(19) United States
(12) Patent Application Publication
(10) Pub. No.: US 2024/0299212 A1
(43) Pub. Date: Sep. 12, 2024

(54) PORTABLE COOLING DEVICE

(71) Applicant: BrainCool AB, Lund (SE)

(72) Inventors: Mohammad Fazel BAKHSHESHI, Lund (SE); Hakan HÜMMET, Malmö (SE); Trung TRAN, Lund (SE)

(73) Assignee: BrainCool AB, Lund (SE)

(21) Appl. No.: 18/547,675

(22) PCT Filed: Feb. 23, 2022
(86) PCT No.: PCT/EP2022/054562

§ 371 Date: Aug. 23, 2023

(30) Foreign Application Priority Data
Feb. 24, 2021 (SE) 2150194-5

(51) Int. Cl. A61F 7/12; A61F 7/00

(52) U.S. Cl. CPC A61F 7/12; A61F 7/0085; A61F 2007/0064; A61F 2007/126

(57) ABSTRACT

A portable cooling device (100) for administering a coolant (133) to a bodily cavity (210) of a user (200) is presented. The portable cooling device (100) comprises a flow transducer (110) configured to provide an airflow (113) to the bodily cavity (210) of the user (200), and a pressure transducer (120) configured to inject the coolant (133) into the airflow (113). The flow transducer (110) and the pressure transducer (120) are separate components. A method of controlling the portable cooling device is also presented.

### TECHNICAL FIELD

The present invention relates to devices for administering cooling to cavities of mammals and more precisely to a portable devices for administering cooling to cavities of mammals.

### BACKGROUND

Cooling of a body of a patient in order to cool inner organs of the patient is used quite often within medicine in general, and in emergency services in particular. For instance, in case of trauma or cardiac arrest, loss of blood flow to the brain can cause severe tissue damage but cooling of the brain has been shown to significantly reduce the risk of tissue damage. Hospitals are typically equipped with heavy and bulky equipment to be able to administer cooling to patients.

The sooner a patient comes under cooling therapy, the lower the risk of tissue damage. This is why emergency personnel may be equipped to administer cooling in the field. The equipment used by emergency personnel is typically lighter and, to some extent portable. The cooling effect of these devices is typically accomplished by injecting an airflow comprising a coolant into cavities of the patient. The airflow is often delivered from a large, bulky gas container, typically a container of pressurized oxygen gas, and the coolant is injected into the airflow by the pressure of the pressurized container.

One such device is disclosed in U.S. Pat. No. 10,561,527 which is adapted for cooling via a patient's nasopharyngeal cavity. Cooling assemblies include at least one elongate tubular member having first and second lumens, a source of liquid coolant, a gas source communicating with the first lumen, and a switch for alternately connecting the liquid coolant source to the second lumen. The first lumen transports a compressed gas from the gas source and the second lumen transports a volatile liquid.

The product RhinoChill by BrainCool is a portable, battery-powered product for rapid and early medical cooling for treatment of sudden cardiac arrest and stroke. With the help of a catheter, coolant is sprayed into the nostrils of a patient. The results of previous European clinical trials of 200 patients at several centers, PRINCE study published in Circulation 2010, showed that when RhinoChill was used in conjunction with cardiac arrest, the temperature in the brain reached protective levels several hours earlier compared to patients chilled by traditional methods at intensive care units. In addition to this, the PRINCESS Randomized Clinical Trial, NORDBERG, Per et al. Effect of Trans-Nasal Evaporative Intra-arrest Cooling on Functional Neurologic Outcome in Out-of-Hospital Cardiac Arrest in: JAMA. 2019; 321(17):1677-1685, showed that early trans-nasal cooling with RhinoChill provided statistically significant improvement in neurological recovery in patients presenting with ventricular fibrillation, VF, arrest.

Although cold-therapy has been demonstrated to relief pain during migraine attack, the clinical efficacy remains limited. The limited clinical efficacy is due to the current rudimentary cooling approaches available, e.g. cooling gel patches, cooling caps. These have limited efficacy as they fail to effectively reduce the brain blood temperature, and rather cool the skin or scalp. Despite these limited effects, surface cooling is the number one self-care treatment for migraine. Cold therapy in migraine works by cooling the blood passing to the intracranial vessels. As such, cold therapy in migraine needs to penetrate the skull to reach these target vessels recent research has shown that cooling via the nasal cavity, the nasopharyngeal cavity, may significantly reduce the symptoms of patient with migraine. The onset of migraine is often sudden and not something that emergency personnel is expected to respond to.

Further to this, cooling of other internal organs in general via orifices of the human body has been shown to relief pain and speed up recovery associated with various illnesses and injuries.

Consequently, there is a need for a cooling device for suitable for self-treatment of a user. Such a cooling device is preferably reduced in size and convenient and easy to use without specialized equipment or training.

### SUMMARY

An object of the present invention is to provide a new type of cooling device which is improved over prior art and which eliminates or at least mitigates the drawbacks discussed above. More specifically, an object of the invention is to provide a cooling device that is easier to transport and more convenient than the prior art. These objects are achieved by the technique set forth in the appended independent claims with preferred embodiments defined in the dependent claims related thereto.

In a first aspect of the invention, a portable cooling device for administering a coolant to a bodily cavity of a user is presented. The portable cooling device comprises a flow transducer that is configured to provide an airflow to the bodily cavity of the user. Further to this, the portable cooling device comprises a pressure transducer configured to inject the coolant into the airflow, wherein the flow transducer and the pressure transducer are separate components.

In one variant of the portable cooling device, the airflow is provided to the cavity of the user by means of a first tubing. This is beneficial since tunings are cost effective, flexible and may be configured into any suitable shape or form.

In one variant of the portable cooling device, it further comprises a coolant container comprising the coolant. A separate coolant container is beneficial since it allows the refilling and replacing of the container in a cost and logistically efficient manner.

In one variant of the portable cooling device, the coolant container is arranged to be pressurized by the pressure transducer via a second tubing. The coolant container is arranged to inject the coolant into the airflow via a third tubing. This is beneficial since it enable the coolant container to directly inject the coolant without the need of further external components.

In one variant of the portable cooling device, the coolant container further comprises a first terminal connected to the second tubing, and a second terminal connected to the third tubing. This is beneficial since the terminals provide an easy and secure means of connecting and disconnecting either of the tubing from the coolant container.

In one variant of the portable cooling device, the cooling container further comprises a closure comprising at least one of said first terminal and said second terminal. This is very beneficial since e.g. the replacement of the coolant container would not require disconnecting the tubing from the terminals, but rather just removing the coolant container from the closure and replacing it with another coolant container when the coolant needs replenishing.

In one variant of the portable cooling device, the pressure transducer is one or more piezoelectric micro blowers. Piezoelectric micro blowers are very beneficial as they are able to deliver a steady and accurate pressure based on control signals.

In one variant of the portable cooling device, the flow transducer is one or more fans. Fans are very cost effective and easy to come by off the shelf components that are adept at providing an accurate and controllable airflow.

In one variant of the portable cooling device, it further comprises at least one flow sensor arranged to measure the airflow provided to the user. Integrating a flow sensor in the portable cooling device is beneficial as it allows for a more accurate control of the airflow.

In one variant of the portable cooling device, it further comprises at least one pressure sensor arranged to measure a pressure provided by the pressure transducer. Integrating a pressure sensor in the portable cooling device is beneficial as it allows for a more accurate control of the pressure and thereby the amount of coolant injected into the airflow.

In one variant of the portable cooling device, it further comprises at least one controller configured to control the flow transducer to provide a predetermined or configurable airflow and/or configured to control the pressure transducer to provide a predetermined or configurable pressure. A controller is beneficial as it allows accurate and correct control of the cooling administered based on e.g. needs, desires or requirements of the user.

In one variant of the portable cooling device, the controller is configured to control the flow transducer based on measurement data from said at least one flow sensor and/or wherein the controller is configured to control the pressure transducer based on measurement data from said at least one pressure sensor. This is beneficial as it allows accurate and correct control of the cooling administered based on e.g. needs, desires or requirements of the user.

In a second aspect of the invention a method of controlling the portable cooling device is presented. The method comprises controlling the flow transducer to provide an airflow to the user. The flow transducer is controlled independently of the pressure transducer. The method further comprises controlling the pressure transducer to provide a pressure to inject the coolant into the airflow. The pressure transducer is controlled independently of the flow transducer.

In one variant of the method, the step of controlling the flow transducer further comprises acquiring a predetermined or configurable airflow, and acquiring a current airflow from least one flow sensor arranged to measure the airflow. The step of controlling the flow transducer to provide an airflow, is based on a difference between the acquired predetermined or configurable airflow and the acquired current airflow. This is beneficial as it allows an accurate control of the airflow.

In one variant of the method, the step of controlling the pressure transducer further comprises acquiring a predetermined or configurable pressure, and acquiring a current pressure from least one pressure sensor arranged to measure the pressure. The step of controlling the pressure transducer to provide a pressure, is based on a difference between the acquired predetermined or configurable pressure and the acquired current pressure. This is beneficial as it allows an accurate control of the airflow.

### BRIEF DESCRIPTION OF THE DRAWINGS

Embodiments of the invention will be described in the following; references being made to the appended diagrammatical drawings which illustrate non-limiting examples of how the inventive concept can be reduced into practice.

FIG. 1 is a schematic view of a coolant device of the prior art

FIG. 2 is a schematic view of a portable cooling device according to embodiments of the invention.

FIG. 3 is a schematic view of a coolant container according to embodiments of the invention.

FIG. 4 is a partial schematic view of a portable cooling device according to embodiments of the invention.

FIG. 5 is a schematic view of a method of controlling a portable cooling device according to embodiments of the invention.

### DETAILED DESCRIPTION OF EMBODIMENTS

Hereinafter, certain embodiments will be described more fully with reference to the accompanying drawings. The invention may, however, be embodied in many different forms and should not be construed as limited to the embodiments set forth herein; rather, these embodiments are provided by way of example so that this disclosure will be thorough and complete, and will fully convey the scope of the invention, such as it is defined in the appended claims, to those skilled in the art.

The term “coupled” is defined as connected, although not necessarily directly, and not necessarily mechanically. Two or more items that are “coupled” may be integral with each other. The terms “a” and “an” are defined as one or more unless this disclosure explicitly requires otherwise. The terms “substantially,” “approximately,” and “about” are defined as largely, but not necessarily wholly what is specified, as understood by a person of ordinary skill in the art. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a device that “comprises,” “has,” “includes” or “contains” one or more steps possesses those one or more steps, but is not limited to possessing only those one or more steps.

The word transducer, when used throughout this disclosure it to mean a device that is actuated by power from one system and supplies power in another form to a second system. In particular, the first system supplying power is an electrical system and the second system will be defined based on the name of the transducer. A flow transducer is actuated by electrical power and transforms the electrical power to a flow of fluid in the second system. Analogously, a pressure transducer is actuated by electrical power and transforms the electrical power to a, typically increased, atmospheric pressure in the second system.

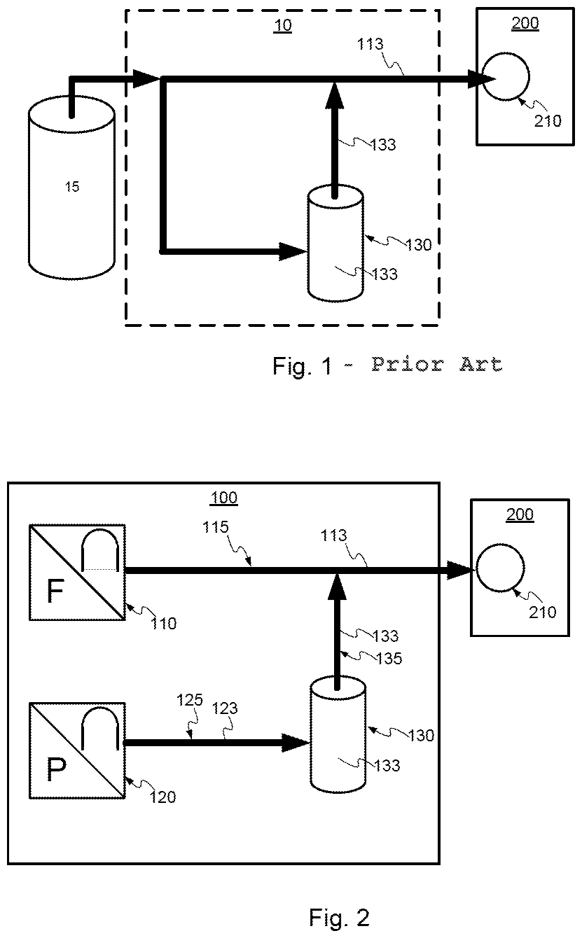

As explained in previous sections, the RhinoChill device is very efficient for medical use in cooling nasopharyngeal cavities of a patient. However, albeit considered portable, the RhinoChill is still too bulky for use in self-treatment. FIG. 1 schematically illustrates a simplified cooling device 10 of the prior art. An external gas source 15 is arranged to supply pressure to, i.e. pressurize, a coolant container 130 to provide an airflow 113 to a bodily cavity 210 of a patient 200. Due to the pressure applied to the coolant container 130, a coolant 133 is injected into the airflow 113 that is supplied to the bodily cavity 210 of the patient 200. The external gas source 15 is typically a container containing pressurized oxygen and such containers are usually large, heavy and not something that is readily sold in stores or pharmacies.

The inventors behind this disclosure have realized that a portable cooling device 100 suitable for self-treatment should be designed to work without the external gas source 15. However, the external gas source 15 both provides pressure for the coolant container 130 and the airflow 113 to the bodily cavity 210 of the patient 200. In order to provide a controlled cooling, it is important that the airflow 113 is substantially constant, but even more important, the pressure applied to the coolant container 130 must be accurately controlled in order to inject the desired amount of coolant 133 into the airflow 113. Further inventive thinking led to the realization that the features of providing an airflow and providing a pressure may be separated and controlled by independent devices, neither one being an external gas container 15. Apart from allowing the removal of the external gas container making the design more convenient, such a designs allows for the airflow 113 to be controlled independently of the coolant 133 and vice versa.

Turning to FIG. 2, a schematic view of a portable cooling device 100 according to embodiments of the invention is shown. The gas container 15 of the prior art is, in the portable cooling device 100, replaced with a separate flow transducer 110 and a separate pressure transducer 120. The flow transducer 110 is configured to provide the airflow 113 to the bodily cavity 210 of the patient 200 or a user 200. The pressure transducer 120 is configured to inject the coolant 133 into the airflow 113. The injection of coolant 133 into the airflow 113 may be accomplished by the pressure transducer 120 pressurizing a coolant container 130 comprising the coolant 133. The coolant container 130 may be internal to the portable cooling device 100, or externally connectable to the portable cooling device 100. More details relating to the coolant container 130 will be given in coming sections.

The flow transducer 110 may be operatively connected to a first tubing 115 into which the airflow 113 is provided by the flow transducer 110. The first tubing 115 is typically terminated in a catheter adapted to the bodily cavity 210 of the user 200. The pressure transducer 120 is operatively connected to the coolant container 130, preferably by means of a second tubing 125. When the pressure transducer 120 is controlled to generate a predetermined or configurable pressure 123, this pressure 123 is transferred to the coolant container 130 via the second tubing 125. The coolant container 130 is operatively connected in fluid communication to the first tubing 115 such that when a pressure 123 above a pressure threshold is subjected to the coolant container 130 via the second tubing 125, coolant 133 is injected into the first tubing 115. The coolant container 130 may be operatively connected to the first tubing 115 by means of a third tubing 135.

Having the portable cooling device 100 configured with the pressure transducer 120 separate from the flow transducer 110, i.e. as separate components 110, 120, removes the requirement of the flow transducer 110 having to accurately and substantially constantly apply pressure to the coolant container 130 in order to inject coolant 133 into the airflow 113. This makes it possible to choose the flow transducer 110 from a wider selection of flow transducers 110. As an example, assume that the external gas container 15 in the cooling device 10 of the prior art is replaced with a commonly available axial fan. Axial fans are cost effective and very good at creating a substantially constant flow of air, but the pressure exerted by such a fan will be greatly depending on the load, i.e. the flow resistance of the bodily cavity 210 of the user 200. Therefore, it is not possible to directly replace the external gas container 15 in the cooling device 10 of the prior art with an axial fan. A centrifugal fan may provide an improved alternative, but although centrifugal fans are better at providing a constant pressure compared to axial fans, they are still not accurate enough. In having the flow transducer 110 independent of the pressure transducer 120, as illustrated in FIG. 2, the slow transducer may be in the form of an of the shelf axial fan. This will greatly decrease the cost of the portable cooling device 100 as the need for pneumatic valves and the like required to control the pressure and flow of fluid from the external gas container 15 is removed. Further to this, fans are easily controlled by electronics means and the flow produced by the fan is linked to the current supplied to the fan. It is also possible to mount two or more fans in series in order to increase the maximum flow that can be produced by the fans and consequently also the dynamic range of the flow, i.e. the difference between a minimum flow provided by the flow transducer 110 and a maximum flow provided by the flow transducer 110.

In one embodiment of the portable cooling device 100, the flow transducer 110 is one or more fans. In a further embodiment, the flow transducer 110 is one or more axial fans. In one embodiment, the flow transducer 110 is a plurality of axial fans.

The pressure transducer 120 of the portable cooling device 100 is preferably in the form of one or more piezoelectric micro blower or piezoelectric micro pump. These piezoelectric micro blowers or piezoelectric micro pumps are very well adapted in providing a well specified flow or pressure. The piezoelectric micro blowers may theoretically be used also as flow transducers 110, but the rate of air flow provided by one micro blower is low compared to that of a fan, and it would require a large number of micro blowers in order to provide the airflow necessary to cool a bodily cavity 210 of a user 200.

In one embodiment of the portable cooling device 100, the flow transducer 110 is one or more piezoelectric micro blower or piezoelectric micro pump. In one embodiment, the flow transducer 110 is one piezoelectric micro blower.

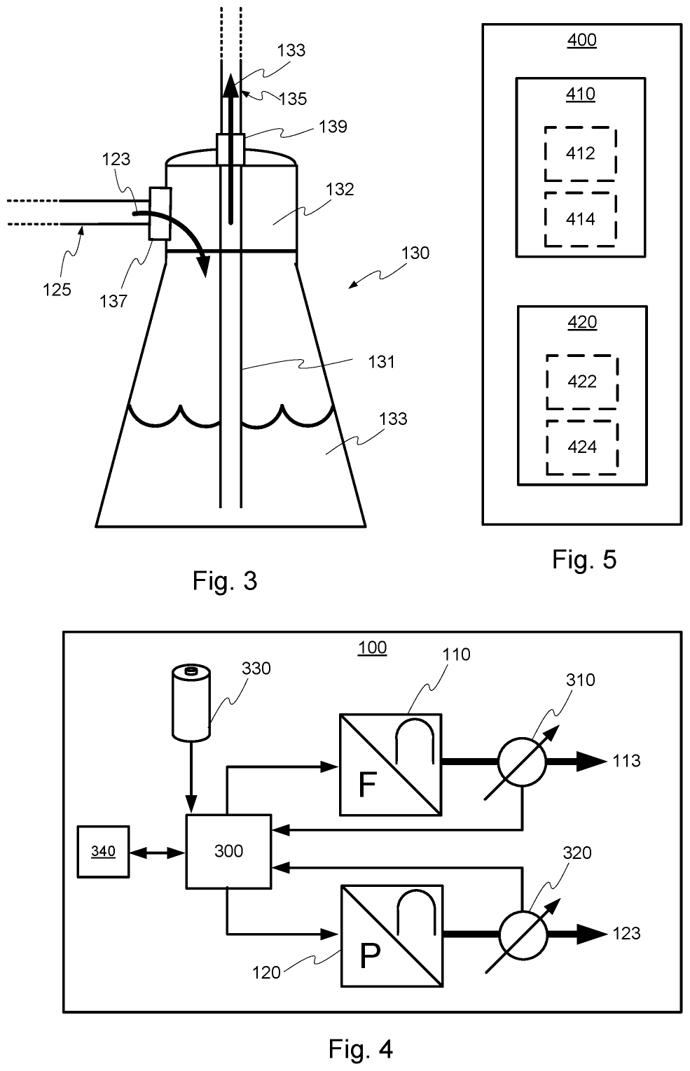

With reference to FIG. 3, one embodiment of the coolant container 130 will be explained. The coolant container 130 comprises a liquid coolant 133, preferably a volatile liquid coolant 133 such as liquid oxygen. The coolant 133 may be any suitable coolant such as a coolant 133 having a boiling point of 38-300° C., preferably a boiling point of 38-200° C., more preferably a boiling point of 60-150° C., more preferably a boiling point of 70-125° C., more preferably a boiling point of 75°−110° C., more preferably a boiling point of 60-70° C. Compounds having suitable characteristics for use herein comprise hydrocarbons, fluorocarbons, perfluorocarbons, and perfluorohydrocarbons. Saline is another example of a substance having suitable characteristics for use herein. As used in this disclosure, the terms “fluorocarbon,” “perfluorocarbon,” and “perfluorohydrocarbon” are synonymous. In addition to containing carbon and fluorine, these compounds may also contain other atoms. In one embodiment, the compounds could contain a heteroatom, such as nitrogen, oxygen, or sulfur, or a halogen, such as bromine or chlorine. These compounds may be linear, branched, or cyclic, saturated or unsaturated, or any combination thereof. The particular choice of coolant is not essential to the invention and the skilled person will, after contemplating the teachings of this disclosure, how to choose a suitable coolant 133.

The coolant 133 is effective both in it having a temperature that is lower than a temperature of the patient, and its volatile properties causes vaporization when the coolant 133 contact the user 200 resulting in an endothermic reaction, further cooling the user. The second tubing 125 is connected to the coolant container 130 such that the pressure generated by the pressure transducer 120 is transferred to an interior of the coolant container 130. The second tubing 125 may be arranged to terminate inside the coolant container 130 but is preferably connected to a side of a first terminal 137 that is external to the pressure container. The first terminal 137 extends to an internal of the pressure container 130 such that the second tubing 125, when connected to a side of the first terminal 137 that is external to the pressure container 130, is in fluid communication with the inside of the pressure container 130. As the internal pressure of the coolant container 130 increases, the coolant 133 exits the coolant container 130 into the third tubing 135 by means of a straw portion 131. The straw portion 131 is a hollow portion that is open at one end which is located such that it typically is submerged in the coolant 133. The straw portion 131 may extend through the coolant container 130 and connect to the third tubing 135, or it may terminate at a side of a second terminal 139 internal to the coolant container 130. The second terminal 139 extends through the coolant container 130 and connects to the third tubing 135 at a side of the second terminal 139 that is external to the coolant container 130.

In one embodiment of the portable cooling device 100, the coolant container 130 comprises a closure 132 in the form of a cap, lid or the like. The closure 132 may be provided with one or both of the first terminal 137 and/or the second terminal 139.

In one embodiment, the closure 132 comprises both the first terminal 137 and the second terminal 139 and the closure 132 is attached to the portable cooling device 100. In this embodiment, the coolant container is connected to the closure 132 and thereby held to the portable cooling device 100 by means of the attachment between the closure 132 and the portable cooling device 100.

Having the one or more of the tubing 137, 139 connected to the closure 132 is beneficial since the user does not have to remove any tubing 125, 135 when changing the coolant container 130. The connecting and disconnecting of tubing 125, 135 will add to the wear of the portable cooling device 100 and increase the risk of e.g. leaks reducing the lifetime of the portable cooling device 100.

In FIG. 3 schematic view of the portable cooling device 100 according to the embodiments is shown. The embodiment of FIG. 3 utilizes one further benefit of having the flow transducer 110 separate from the pressure transducer 120 in that the transducers 110, 120 are independently controlled. Each of the transducers 110, 120 may be controlled by one or more control means 300. This means that pressure 123 and the airflow 113 may be controlled such that e.g. a constant concentration of coolant 123 is injected into the airflow 113 regardless of the flow rate of the airflow 113, the airflow 113 is increased without increasing the coolant 123 injected into the airflow thereby effectively reducing the concentration of coolant 123 in the airflow 113 etc. In one embodiment, that may be combined with any of the other embodiments mentioned, the portable cooling device 100 comprises a control means 300 in the form of a controller 300, or a control means 300 in the form of a controller 300 is operatively connected to the portable cooling device 100. The controller 300 may be configured to control the flow transducer 110 to provide a predetermined or configurable airflow 113 and/or configured to control the pressure transducer 120 to provide a predetermined or configurable pressure 123.

In order to more accurately control the airflow 113, the portable cooling device 100 may, in optional embodiments, be provided with at least one flow sensor 310 arranged to measure the airflow 113 provided to the user 200. The one or more flow sensor 310 may be arranged at any suitable location along the first tubing 115. The use of flow sensors 310 is very beneficial since it, in addition to enabling more accurate control of the airflow 113, allows for the detection of any changes in airflow 113 that may occur due to e.g. the blockage of or withdrawal of the catheter at the end of the first tuning 115 from the bodily cavity 210 of the user 200. The withdrawal of the catheter or the blockage of the catheter will change the flow resistance of the first tubing 115 and result in a change of the airflow. In some embodiments, a first flow sensor 310 is located in the first tubing 115 between the third tubing 135 and the flow transducer 110, and a second flow sensor 310 is located in the first tubing 115 between the third tubing and the user 200. Such an arrangement is beneficial since it allows the detection of any leaks and a difference in detected airflow by first and the second flow sensors 310 may, be used to determine if the coolant container 130 needs refilling or replacing. In one embodiment of the portable cooling device 100, a flow sensor 310 is arranged in the third tubing 135 to detect the flow of coolant 133 that is injected into the airflow 133.

In order to more accurately control the pressure 123, the portable cooling device 100 may, in optional embodiments, be provided with at least one pressure sensor 320 arranged to measure the pressure 123 provided to the coolant container 130. The one or more pressure sensors 320 may be arranged at any suitable location along the second tubing 125 or in the coolant container 130. The use of pressure sensors 320 is very beneficial since it, in addition to enabling more accurate control of the pressure 123, allows for the detection of any changes in pressure 123 that may occur due to e.g. a level of coolant 133 in the coolant container 130 is becoming low or a leakage between the pressure transducer 120 and the coolant container 130. If the level of coolant 133 in the coolant container 130 is reduced such that e.g. the straw portion 131 is no longer submerged in the coolant 133 the pressure 123 will change and this is detectable by the pressure sensor 320.

The use of the different sensors 310, 320 as explained above is, as the skilled person understands after reading this disclosure, possible to combine with any suitable embodiment of the portable cooling device 100. It is further possible to combine one or more pressure sensors 320 with one or more flow sensors 310 at different locations of the path of the airflow 113 and/or the extension of the pressure 123.

In embodiments where the portable cooling device 100 comprises a controller 300, any flow sensor 310 and/or pressure sensor 320 comprised in the portable cooling device 100, is preferably operatively connected to, and arranged to provide measurement data to, the controller 300.

In order to further increase the portability of the portable cooling device 100 and make it independent of a mains outlet, the portable cooling device 100 may be provided with a battery 330. The battery 330 may be arranged to power at least one of the flow transducer 110, the pressure transducer 120, the controller 300, the pressure sensor 320 and/or the flow sensor 310. The battery 330 may be any suitable battery 330 but is preferably a rechargeable battery 330. The portable cooling device 100 may comprise charging electronics such that the rechargeable battery is charged in the portable cooling device 100, alternatively or additionally, an external charger may be provided such that the rechargeable battery is charged externally to the portable cooling device 100. The rechargeable battery 330 is preferably a lithium-type battery which is beneficial due to their high energy density, relatively low self-discharge and low maintenance.

In order to provide feedback to the user 200, and/or to allow the user to control the portable cooling device 100, the portable cooling device 100 may be provided with an interface unit 340. The interface unit 340 may be any suitable unit providing an interface to the user 200. The interface unit 340 may be configured to allow the user 200 to control the airflow 113 and/or the pressure 123. It may additionally or alternatively be configured to provide feedback to the user 200 regarding e.g. a current airflow 113, a current pressure 123, a time the device has been used, charged status of the battery 330 etc. The interface unit 340 may be in the form of a software running on a portable electronics equipment that is wirelessly linked to the portable cooling device 100.

Further to the inventive portable cooling device 100, the inventors have invented a method 400 of controlling the portable cooling device 100. FIG. 5 illustrates a schematic flow chart of the method 400 for controlling the portable cooling device any of the before mentioned embodiments.

The method 400 comprises controlling 410 the flow transducer 110 to provide the airflow 113 to the user 200. Optionally, the controlling 410 or the flow transducer 110 may be based on a current airflow 113 measured by one or more flow sensors 310. In such an embodiment, the step of controlling the flow transducer further comprise acquiring 414 a current airflow 113 the one or more flow sensors 310 comprised in the portable cooling device 100. The controlling 410 may be in the form of a control loop wherein the acquired 414 airflow 113 is compared to a predetermined or configurable airflow. The method 400 may comprise the step of acquiring 412 the predetermined or configurable airflow. This may be acquired 412 form e.g. the interface unit 340 or retrieved from a volatile or non-volatile memory of the portable cooling device 100. The control loop of the air flow 113 may be any suitable control loop comprising product, derivative and/or integral parts as are well known in the art.

The method 400 further comprises controlling 420 the pressure transducer 120 to provide the pressure 123 to inject the coolant 133 into the airflow 113. Optionally, the controlling 420 or the pressure transducer 120 may be based on a current pressure 123 measured by one or more pressure sensors 320. In such an embodiment, the step of controlling 420 the pressure transducer further comprise acquiring 424 a current pressure 123 from the one or more pressure sensors 320 comprised in the portable cooling device 100. The controlling 420 may be in the form of a control loop wherein the acquired 424 pressure 123 is compared to a predetermined or configurable pressure. The method 400 may comprise the step of acquiring 422 the predetermined or configurable pressure. This may be acquired 422 form e.g. the interface unit 340 or retrieved from a volatile or non-volatile memory of the portable cooling device 100. The control loop of the pressure 123 may be any suitable control loop comprising product, derivative and/or integral parts as are well known in the art.

In embodiments of the method 400, it may further comprise a control step control the flow transducer 110 and/or the pressure transducer 120 to provide a constant concentration of coolant 133 to the cavity 210 of the user 200 regardless of the airflow 113.

It should be mentioned that the method 400 may very well be performed by the controller 300. Further to this, the method 400 may further comprise obtaining control parameters, e.g. a wanted airflow, a wanted pressure and/or a wanted concertation of coolant in the airflow from the interface unit 340. The method 400 may further comprise showing information regarding a current status of any parameter relating to the portable cooling device 100 and/or the method 400 to the user, e.g. on a display device or the interface device 340.

1. A portable cooling device for administering a coolant to a bodily cavity of a user, the portable cooling device comprising:
a flow transducer configured to provide an airflow to the bodily cavity of the user; and
a pressure transducer configured to inject the coolant into the airflow;
wherein the flow transducer and the pressure transducer are separate components.

2. The portable cooling device of claim 1, wherein the airflow is provided to the cavity of the user by means of a first tubing.

3. The portable cooling device of claim 1, further comprising a coolant container comprising the coolant.

4. The portable cooling device of claim 3, wherein the coolant container is arranged to be pressurized by the pressure transducer via a second tubing; and
wherein the coolant container is arranged to inject the coolant into the airflow via a third tubing.

5. The portable cooling device of claim 4, wherein the coolant container further comprises a first terminal connected to the second tubing, and a second terminal connected to the third tubing.

6. The portable cooling device of claim 5 wherein the cooling container further comprises a closure having at least one of the first terminal and the second terminal.

7. The portable cooling device of claim 1, wherein the pressure transducer is one or more piezoelectric micro blowers.

8. The portable cooling device of claim 1, wherein the flow transducer is one or more fans.

9. The portable cooling device of claim 1, further comprising at least one flow sensor arranged to measure the airflow provided to the user.

10. The portable cooling device of claim 1, further comprising at least one pressure sensor arranged to measure a pressure provided by the pressure transducer.

11. The portable cooling device of claim 1, further comprising at least one controller configured to control the flow transducer to provide a predetermined or configurable airflow and/or configured to control the pressure transducer to provide a predetermined or configurable pressure.

12. The portable cooling device of claim 11, wherein the controller is configured to control the flow transducer based on measurement data from at least one flow sensor arranged to measure the airflow provided to the user; and/or
wherein the controller is configured to control the pressure transducer based on measurement data from at least one pressure sensor arranged to measure a pressure provided by the pressure transducer.

13. A method of controlling the portable cooling device of claim 1, the method comprising:
controlling the flow transducer to provide an airflow to the user, wherein the flow transducer is controlled independently of the pressure transducer; and
controlling the pressure transducer to provide a pressure to inject the coolant into the airflow, wherein the pressure transducer is controlled independently of the flow transducer.

14. The method of claim 13, wherein controlling the flow transducer further comprises:
acquiring a predetermined or configurable airflow;
acquiring a current airflow from at least one flow sensor arranged to measure the airflow;
wherein controlling the flow transducer to provide an airflow, is based on a difference between the acquired predetermined or configurable airflow and the acquired current airflow.

15. The method of claim 13, wherein controlling the pressure transducer further comprises:
acquiring a predetermined or configurable pressure;
acquiring a current pressure from at least one pressure sensor arranged to measure the pressure;
wherein controlling the pressure transducer to provide a pressure, is based on a difference between the acquired predetermined or configurable pressure and the acquired current pressure.
